(19) United States
(12) Patent Application Publication
(10) Pub. No.: US 2013/0004968 A1
(43) Pub. Date: Jan. 3, 2013

(54) Sepsis blood biomarker system

(76) Inventor: Robert Webber, Las Vegas, NV (US)

(21) Appl. No.: 13/200,233

(22) Filed: Sep. 20, 2011

Related U.S. Application Data
(60) Provisional application No. 61/403,919, filed on Sep. 22, 2010.

(51) Int. Cl. G01N 21/76; G01N 33/577; C12Q 1/26

(52) U.S. Cl. 435/7.9; 435/25; 435/7.1

(57) ABSTRACT

A panel of blood biomarkers for assessing a sepsis condition utilizing an iNOS indicator in combination with one or more indicators of patient predisposition to becoming septic, the existence of organ damage, or the worsening or recovering from a sepsis episode.

### CROSS-REFERENCE TO RELATED APPLICATION

The application claims the benefit, under 35 U.S.C. §119(e), of U.S. Provisional Patent Application No. 61/403,919, filed on 22 Sep. 2010, which is incorporated herein, by reference, in its entirety.

### BACKGROUND OF THE INVENTION

The present invention relates to a novel and useful panel of biomarkers indicating the predisposition, onset, progression of sepsis, as well as the existence of organ damage due to the sepsis pathology i.e. sepsis condition.

At least 7 million patients per year enter into the early stages of the sepsis pathology, a medical condition named systemic inflammatory response syndrome (SIRS), which will lead to more than 250,000 deaths per year in the USA and more than 750,000 worldwide. It is predicted that larger numbers of persons will develop SIRS as the population ages. Sepsis develops from a variety of bacterial and fungal sources stemming from the patient's inability to fight infection, and is commonly acquired while recovering from severe injuries and surgery in hospitals.

Reference is made to Levenson, D (2008) “The Quest For Faster Sepsis Diagnosis” Clinical Lab News, 34 #1: 1-5; and Bone, R (1996) “Why Sepsis Trials Fail” JAMA, 276#7: 565-566 which describe the need for the early diagnosis of the enormous sepsis pathology to provide early patient treatment and to rearrange the costs of treatment. There is also a need for supplemental tests for the sepsis pathology. Namely, supplemental tests are needed to provide data:

1. To differentiate between patients who are suspected of becoming septic and who will not develop sepsis and those patients who are suspected of becoming septic and who will become septic, severely septic or suffer from septic shock.

2. To determine the susceptibility of an individual patient to becoming septic.

3. To place an individual patient's current status as “very early”, “early”, or “mid-stage” in an episode of sepsis; and

4. Regarding the probability that an individual patient's condition is expected to deteriorate or to improve.

In addition, such tests are necessary to form a decision tree, allowing the attending physician to make critically important treatment decisions for their patient.

Current laboratory culture procedures for diagnosing sepsis suffer from a number of problems:

1. They are slow. The first answer using current culture procedures is obtained only after 24 hours of culture, and an absolute answer requires a minimum of 48 hours of culture.

2. Blood culture only yields positive results (i.e. sepsis is present) in approximately 28% of patients who become septic, Thus, over 70% of the patients do not yield positive blood cultures. Sands, K E, et al (1997) “Epidemiology Of Sepsis In The United States From 1979 to 2000” N Engl J Med, 348: 1546-1554; and Martin, C S, et al (2003) “Epidemiology Of Sepsis In 8 Academic Medical Centers” JANA, 278: 234-240.

Potential biochemical markers of sepsis and severe sepsis, such as TNFalpha, IL-1beta, IL-6, nitrate/nitrite, lactate, procalcitonin, and many others, have been evaluated by various investigators for their predictive value. Only three have been reported to be useful. Two groups of investigators have reported that a very high level of IL-6 (greater than 1000 pg/mL) is linked to a hyperinflammatory condition and is predictive of a poor outcome in septic patients receiving a neutralizing TNFalpha monoclonal antibody.

Increased levels of procalcitonin have been measured in patients with SIRS, sepsis and severe sepsis. The source of the circulating procalcitonin derives from activated parenchymal cells, but its increased presence in blood is not associated with a circulating increase in either calcitonin or calcitonin gene related peptide, which are the two “normal” products obtained from the processing of procalcitonin. In the Levenson. D. reference, supra, it was reported that the procalcitonin assay (PCT) has been FDA approved for diagnosing sepsis in ICUs, but its use has not resulted in decreased morbidity or mortality.

An increase in the concentration of nitrate/nitrite in blood has been shown to be associated with sepsis and may be predictive of a poor outcome. The source of the increased nitrate/nitrite level has been postulated to be due to the induction of iNOS. The induction of iNOS has been shown not only to occur in the presence of Gram-negative bacteria, but also with Gram-positive bacteria and fungal infections. A test for plasma endotoxin (lipopolysaccharide or LPS) has also been approved by the FDA for the diagnosis of sepsis, the endotoxin activity assay (EAA test): However, as with the procalcitoinin PCT assay, its use has not proven to decrease morbidity or mortality, per the Levenson reference, supra.

A molecular diagnostic PCR test (Roche PCR) has been employed in Europe for a number of years, but the Roche PCR test has not reduced the incidents or severity of the sepsis pathology.

Neither the PCT test, the Endotoxin EAA test, nor the Roche PCR based test has proven satisfactory for the early diagnosis of sepsis, even though all three tests are FDA approved. The Levenson reference, supra, concludes that none of the currently FDA approved diagnosis procedures work well.

As heretofore stated, sepsis almost always starts with a bacterial or fungal infection, but the pathology results from an individual patient's hyperinflammatory response to cell wall components from dead micro-organisms, which are produced when the body attempts to fight off the infection where such micro-organisms are killed. The dead micro-organism release a portion of its cell wall into the blood sets off the “cytokine storm”. In some individuals this series of events ultimately leads to the pathology known as sepsis. The article of Sands K E, Martin G S, supra, and another article; Kohn L T, Corrigan J M, Donaldson M S, editors, (2000) “To Err Is Human: Building A Safer Health System”. Washington (D.C.): National Academies Press, pp. 1-287, describe such sepsis pathology.

The concept of “sterile” sepsis exists, but has been very difficult to prove. “Sterile sepsis” has been postulated to be initiated by necrotic cells from major trauma which release their intracellular contents, particularly mitochondrial nucleic acids and proteins, into the circulatory system and, thereby, trigger the “cytokine storm”. Irrespective of the mode of induction, the “cytokine storm” leads to the expression of iNOS. Further, when a cell is induced to express iNOS, it is destined to die by programmed cell death (apoptosis). To understand the onset of the sepsis pathology, one needs to understand how the apoptotic process malfunctions in sepsis. Cells induced, by the “cytokine storm”, to express iNOS are normally destined to die by apoptosis and to be scavenged by macrophages. Cells to be scavenged by macrophages mark themselves with “eat me” signals by expressing new receptors on their exterior cell membrane and by transferring phosphatidyl serine from the interior side of the cellular membrane to the exterior side of the lipid bilayer. These are the “eat me” signals for a macrophage to phagocytose the apoptotic cell, as-well-as apoptotic bodies or other microvesicles shed from the cell. The result of this normal scavenging process is that none of an apoptotic cell's components is released.

U.S. Pat. Nos. 6,531,578; and 7,188,904 and articles by Webber, R. J. and Dunnebacke, T. H. (2003) “Inducible Nitric Oxide Synthase Is An Early Plasma Biomarker For The Onset Of Sepis” 43^{rd} Inter. Conf. Antimicrob. Agents Chemothem. (Webber I); Webber, R. J., et al (2005) “Development, Characterization, And Epitope Mapping Of A Panel Of Twenty-Four Monoclonal Antibodies Specific For Human Inducible Nitric Oxide Synthase Hybridoma”, 24:6-13. (Webber II); Gambin, M H. et al (2007) “Platelet-derived Exosomes Induce Endothelial Cell Apoptosis Through Peroxynitrite Generation: Experimental Evidence For A Novel Mechanism Of Septic Vascular Dysfunction” Critical Care 2007, 11: R107; Azevedo LCP, et al (2007) “Platelet-derived Exosomes From Septic Shock Patients Induce Myocardial Dysfunction” Critical Care 2007, 11:R120; Mortaza S, et al (2009) “Detrimental Hemodynamic And Inflammatory Effects Of Microparticles Originating From Septic Rats” Critical Care Med, 37#6: 2045-2060; Webber R. J., Webber, D. S., and Dixon T. H. (2005) “Improved Therapeutic Agent For iNOS Generating Illness” PCT Application WO2005/120569 Filed May 19, 2005 based Upon U.S. patent application Ser. No. 11/129,452 (Webber III); Webber, R. J., Dunnebacke, T. H., And Webber, D. S. (2006) “Neutralization In Vivo Of Particulate iNOS With Humanized Anti-iNOS mAbs Rescues Mice From Death By Sepis” 46^{th} Inter. Conf. Antimicrob. Agents Chemothem. (Webber IV), all address the problems associated with the normal scavenging process going awry. The data in these references convincingly demonstrate that what triggers the sepsis pathology is aberrant apoptosis in which macrophages do not properly scavenge induced, iNOS-containing apoptotic cells, since either:

1. The iNOS-containing apoptotic cells do not mark themselves correctly with the “eat me” signs;

2. Macrophages do not recognize the “eat me” markings on the iNOS-containing apoptotic cells correctly, or;

3. A local depletion of macrophages occurs and other macrophages can not be recruited to a site fast enough to scavenge the iNOS-containing apoptotic cell.

It has also been indicated in Webber III and Webber IV, supra, that unscavenged apoptotic cells undergo secondary necrosis. That is, the cells swell, burst, and release their contents into the circulatory system. The data of the prior cited references clearly show that the rupture of induced, iNOS-containing, apoptotic cells, which undergo aberrant apoptosis and die by secondary necrosis, instead of programmed cell death, result in the release of iNOS into the circulatory system. Data has proved this mechanism only occurs in septic patients since the presence of iNOS can be detected and measured by sandwich EIA and by Western blot analysis exclusively in plasma samples obtained from septic patients or patients who will become septic in the next 24-72 hours. Such result has been demonstrated in specimens obtained from more than 100 different patients susceptible to becoming septic. Recently, three independent groups of investigators have confirmed these findings. Gambin et al, supra, reported finding iNOS in plasma samples from all septic ICU patients they tested, but not in plasma from prior cited normal healthy volunteers or non-septic ICU patients. Further, the Gambin et al reference, supra, demonstrated that the iNOS in plasma was contained in microvesicles (MVs). Azevedo et al, supra, extended these findings, reporting detecting iNOS in the plasma of every septic ICU patient tested, but again, not in plasma from healthy control or non-septic ICU patients. As in prior cited Gambin et al, the iNOS in plasma was contained in circulating microvesicles. More recently, Mortaza et al, supra, induced sepsis in rats by cecal ligation and puncture which resulted in poly-microbial peritonitis. Mortaza, supra, isolated the circulating MVs from normal rats, sham operated rats, and the septic rats with peritonitis; and then dosed healthy rats with the purified MVs. The MVs isolated from normal and sham operated rats had no effect when administered to the healthy recipient rats. However, the MVs isolated from the septic rats with peritonitis caused hypotension in the healthy recipient rats due to the over production of NO by iNOS translocated by the MVs to the aorta and heart (and probably other locations). This ultimately led to hemodynamic collapse in the recipient animals (many of the rats died of hemodynamic collapse before the end of the study period). To summarize, the combined data of these references demonstrate that what occurs in sepsis is aberrant apoptosis which leads:

[1] To secondary necrosis of cells induced to produce iNOS,

[2] To the release into the circulatory system of iNOS, including MV-associated iNOS, and

[3] To, ultimately, the life-threatening sepsis cascade. Thus, a breakdown in the normal scavenging process of induced, iNOS-containing apoptotic cells leads to the sepsis cascade. In other words, the iNOS-containing apoptotic cells are not properly scavenged by macrophages: instead they undergo secondary necrosis, swell, burst, and release their cellular contents into the circulatory system, including plasma iNOS.

As shown by Gambin et al, Supra, Azevedo et al, Supra Mortaza et al, Supra, U.S. Pat. Nos. 6,531,578, and 7,198,904, Webber I, II, III and IV, supra, it is the cellular and tissue damage to the heart, lungs, kidneys, and other organs that results from the circulating MV-associated iNOS which initiates the sepsis cascade. As long as the iNOS remains in the circulatory system, it is an inactive enzyme, because two of its required cofactors (NADPH and tetrahydrobiopterin) are not present in plasma. However, when the circulating microvesicles lodge onto the exterior membrane of cells at distal sites, the two lipid bilayers fuse, and the contents of the microvesicles are internalized into the “receiver” cell. These processes have been best described for microparticles and microvesicles released from platelets, leukocytes and endothelial cells in the following articles: Ratajczak J, et al (2006) “Membrane-derived Microvesicles: Important And Underappreciated Mediators Of Cell-to-cell Communication” Leukemia, 20: 1487-1495; Ardoin S P (2007) “The Role Of Microparticles In Inflammation And Thrombosis” Scand J Immun, 66: 159-165; Lynch S F, et al (2007) “Plasma Microparticles And Vascular Disorders” Brit J. Haemotol, 137:36-48; Distler J H W, et al (2005) “The Release Of Microparticles By Apoptotic Cells And Their Effects On Macrophage Apoptosis,” 10:731-741; Majka M, et al (2007) “Evidence That Platelet-derived Microvesicles May Transfer Platelet-specific Immunoreactive Antigens To The Surface Of Endothelial Cells And CD34+ Hematopoietic Stem/Progenitor Cells—Implication For The Pathogenesis Of Immune Thrombocytopenias” Folia Histochem et Cytobiol, 45: 27-32; Valadi H, et al (2007) “Exosome-mediated Transfer Of mRNAs And MicroRNAs Is A Novel Mechanism Of Genetic Exchange Between Cells” Nature Cell Biol, 9#1: 654-659. Once an iNOS-containing microvesicle is intercalated into a “receiver” cell, the MV-associated iNOS becomes an active enzyme, since it possesses all its required substrates and cofactors. However, the iNOS enzyme now becomes a component of a cell that has never been induced, iNOS enzyme is in an inappropriate location, and the host “receiver” cell is out of normal cellular regulation. Once inside a “receiver” cell and active, the iNOS enzyme produces toxic quantities of nitric oxide that results in damage to and/or the death of the “receiver” cell: damage to cardiomyocytes leads to hemodynamic collapse (Azevedo LCP and Mortaza, S, supra); and damage to the lungs results in pulmonary dysfunction. Also, damage to the cells that make up the blood-brain barrier, the tight junctions of the intestine, the glomerular endothelial cells of the kidney, and the capillary beds of the circulatory system results in the formation of a microperforation through which leakage can occur as described in the following articles: Han X, et al (2004) “Increased iNOS Activity Is Essential For Pulmonary Epithelial Tight Junction Dysfunction In Endotoxemic Mice” Am J Physiol Lung Cell Mol Physiol, 286: L259-267; Han X, et al (2004) “Increased iNOS Activity Is Essential For Hepatic Epithelial Tight Junction Dysfunction In Endotoxemic Mice” Am J Physiol Gastrointest Liver Physiol, 286: G126-G136; Han X, et al (2004) “Increased iNOS Activity Is Essential For Intestinal Epithelial Tight Junction Dysfunction in Endotoxemic Mice” Shock, 21#3:261-270.

U.S. Pat. Nos. 6,531,578 and 7,198,904 describe a superior in vitro diagnostic (IVD) test for human iNOS, utilizing a panel of contacting monoclonal antibodies and these United States patents are incorporated by reference, herein, in their entirety. iNOS, an intracellular enzyme, is a plasma biomarker that is only found in septic patients or patients who will become septic in the next 24-72 hours. iNOS is not normally present in the blood circulation, but has been found to appear in plasma one to three days before the physiological symptoms of sepsis appear. This IVD test can differentiate between trauma patients in an ICU who will not become septic and those trauma patients in an ICU who will deteriorate into sepsis with organ dysfunction, or septic shock with multiple organ failure.

There is a need for additional and more effective IVD tests to provide additional vital information to the attending physicians, which should allow them to assess an individual patient's septic conditions, such as the patient's susceptibility to becoming septic, the patient's current septic status, and the risk that the patient may progress down the septic pathway.

### SUMMARY OF THE INVENTION

In accordance with the present invention a panel of IVD tests supplementing known tests for showing iNOS in the plasma to assess sepsis conditions in a human subject is herein provided. Such panel of accurate and reliable blood biomarker IVD tests are used to:

1. Differentiate between critically ill patients with inflammation who will not develop sepsis and those critically ill patients who will become septic;

2. Determine the susceptibility of an individual patient to becoming septic;

3. Stage an individual patient's current state as “very early-”, “early-”, or “mid-stage” in an episode of sepsis; and

4. Indicate if an individual patient's condition is expected to deteriorate or to improve.

This panel of supplemental IVD tests represents an enormous medical breakthrough and a major medical advance in the fight against the sepsis pathology. Such a panel of blood tests allows the aggressive application of current Standard of Care therapies earlier, a lifesaving step. It should be realized that the panel of blood biomarkers is intended to be employed in conjunction with the iNOS IVD test found in U.S. Pat. Nos. 6,581,578 and 7,198,904.

Such supplemental tests include measurements of a blood biomarkers, such as lactadherin and like entities, to determine the predisposition of a patient becoming septic. Also, during the course of an episode of sepsis, the change in the blood level of lactadherin will indicate the improvement or worsening of a patient's condition.

Also, to confirm the onset of sepsis, tests are included for measuring the pro-forms of mature inflammatory cytokines such as Pro-IL-1β, Pro-IL-18, and Pro-II-33. Such pro-cytokines reinforce the early detection of plasma iNOS.

Further, other blood biomarkers enzymes that show aberrant apoptosis/secondary necrosis are also found to be useful.

Specifically, cyclooxygenase 2 (COX-2) and hemeoxygenase-1 (HO-1) have been identified as fulfilling this role.

Measurements of Reg 1α (aka pancreatic stone peptide) (PSP) L-Lactate, and the like are employed to determine organ damage at a very early time. Again, increases in the level of Reg 1α and the like, indicate increased organ damage associated with the sepsis pathology.

Plasma/serum biomarkers showing continuing and or increasing inflammation, such as, TNF alpha, IL-1beta, and IL-6 may be used. High levels of these cytokines indicate a worsening of a patient's condition.

Anti-inflammatory biomarkers may also be employed to show patient improvement. For example, high levels of IL-8 and IL-10 would point to patient's recovery.

Blood biomarkers of primary and secondary necrosis are also utilized in the present invention. Plasma/serum proteins or protein complexes such as LDH and cytochrome C (primary and secondary necrosis) show worsening patient conditions when found at high levels.

An decision algorithm of the above disclosed biomarkers has been formed to provide definitive basis for sepsis information to permit an attending physician to decide a course of treatment for a patient.

It may be apparent that a novel and useful panel of biomarkers for use in determining patient status has been hereinabove described.

It is therefore an object of the present invention to provide an accurate and reliable panel of IVD tests that can differentiate between critically ill patients who are not progressing into sepsis and those critically ill patients who are developing sepsis in conjunction with an accurate test for identifying plasma iNOS.

Another object of the present invention is to provide a panel of IVD test that can furnish valuable diagnostic information regarding a patient's susceptibility to sepsis and identify the stage of the septic pathway at which such patient lies.

Another object of the present invention is to provide a panel of IVD tests that have the potential to save lives of patient in a septic condition.

Yet another object of the present invention is to provide a panel of IVD tests as a supplement to tests detecting the onset of a septic condition.

Another object of the present invention is to provide a panel of IVD tests that would significantly reduce the huge long term cost of treating individuals who survive an episode of sepsis, severe sepsis, or septic shock.

A further object of the present invention is to provide a panel of IVD tests which fulfill a clinical laboratory need by providing critical prognostic, diagnostic, and on-going monitoring information to the physicians who are treating these patients, via a decision tree.

Another object of the present invention is to provide a decision tree algorithm or system based upon serum/plasma biomarkers that assists attending physicians in deciding upon treatment for their patients for sepsis conditions.

The invention possesses other objects and advantages which will become apparent as the specification continues.

### BRIEF DESCRIPTION OF THE SEVERAL VIEWS OF THE DRAWING

FIG. 1 is a flow diagram depicting a decision tree employing the tests of the present invention having a positive iNOS EIA test.

FIG. 2 is a flow diagram depicting a decision tree employing the tests of the present invention having a negative iNOS EIA test.

FIG. 3 is a view of Table 2 showing the analysis of data obtained from trauma patients and healthy individuals, with respect to heart, lung, or kidney dysfunction linked to the sepsis pathology.

FIG. 4 is a view of Table 3 indicating the correlation of plasma components to other components in a clinical trial.

FIG. 5 is a view of two scatter diagrams illustrating the plasma levels of iNOS and procalcitonin for the SIRS/sepsis pathology in trauma patients.

FIG. 6 is a graph depicting the levels of iNOS plasma samples taken from multiple human subjects.

FIG. 7 is a graph depicting the levels of lactadherin in plasma samples taken from multiple human subjects.

FIG. 8 is a graph depicting the concentrations of Pro-IL-1β in plasma samples from multiple human subjects.

FIG. 9 is a graph depicting the levels of Pro-IL-18 in plasma samples taken from multiple human subjects.

FIG. 10 is a graph depicting the levels of Pro-IL-33 in plasma samples taken from multiple human subjects.

FIG. 11 is a graph depicting the concentrations of COX-2 in plasma samples taken from multiple human subjects.

FIG. 12 is a graph depicting the levels of Heme Oxygenase-1 (HO-1) in plasma samples taken from multiple human subjects.

FIG. 13 is a graph depicting the levels of Reg 1α in plasma samples taken from multiple human subjects.

FIG. 14 is a graph depicting the concentrations of CRP in plasma samples taken from multiple human subjects.

For a better understanding of the invention reference is made to the following detailed description of the preferred embodiments of the invention which should be taken in conjunction with the above described drawings.

### DETAILED DESCRIPTION OF THE PREFERRED EMBODIMENTS OF THE INVENTION

Various aspects of the present invention will evolve from the following detailed description of the preferred embodiments thereof which should be referenced to the prior described drawings.

A panel of preferred blood biomarkers used in conjunction with the diagnostic test for determining the presence of iNOS described in U.S. Pat. Nos. 6,531,578 and 7,198,904 are herein provided:

An Indicator of a patient's predisposition to sepsis is useful, for example:

Lactadherin (also known as milk fat globulin epidermal growth factor factor-8 and as BA-46) is a plasma/serum protein that acts as a bridging molecule between the exterior receptors on macrophages and other scavenger cells to the phosphatidyl serine moieties on the exterior of apoptotic cells. Low blood levels of lactadherin predispose an individual to become septic since the scavenging of induced apoptotic cells is reduced. If an individual's blood level of lactadherin is below normal, that person will be predisposed to become septic and, if, during the course of an episode of sepsis, the blood level of lactadherin decreases (which will reduce the clearance of induced apoptotic cells) a patient's condition will likely worsen. If the blood level is low and starts to increase, the patient will likely improve.

Additional biomarkers are also important in prognosticating/diagnosing the onset of the sepsis pathology. None of these biomarkers will likely be specific for the sepsis pathology, since such biomarkers can also be released into the blood under other conditions where aberrant apoptosis turns into secondary necrosis. Each of these is the “Pro-form” of a mature inflammatory cytokine. Each pro-form is biosynthesized as an inactive pro-cytokine that is later processed under various conditions by very specific proteases which cleave off the “Pro” sequence at the amino terminus to yield the mature cytokine. The cleavage and activation to the mature cytokine are usually coordinated with cellular secretion. However, cells, induced to produce iNOS (which also leads to the expression of these Pro-cytokines), subsequently become secondarily necrotic, swell, burst and thereby release iNOS into the circulation, will also release these Pro-cytokines (as the “pro-form” not the processed mature form of the cytokine). Thus, the detection and measurement of these “Pro-cytokines”, rather than the mature cytokines, mirror the early detection of iNOS that is to say, such “pro-cytokines confirm that cells induced to produce iNOS are undergoing aberrant apoptosis/secondary necrosis and, in conjunction with a positive iNOS result from the plasma iNOS test of U.S. Pat. No. 6,531,578, or 7,198,904 confirm that the patient is entering the sepsis pathology, for example:

a. The Pro-Interleukin-1β (Pro-IL-1β) assay concerns Pro-IL-1β which is a 31 kDa, 269 amino acid long protein precursor of the mature IL-1β. Pro-IL-1β is synthesized in response to most micro-organisms, to the cell wall components of dead micro-organisms, and to other pro-inflammatory stimuli, such as TNFα and INFγ. There is a dissociation of expression and activation with independent cellular regulation of transcription and translation and of proteolytic cleavage of the precursor to the active mature cytokine. Pro-IL-1β has no IL-1β bioactivity, and is compartmentalized into the cytoplasm prior to cellular secretion. Secretion from the cell is normally coupled to cleavage of Pro-IL-1β into mature, active IL-1β. Thus, extracellular IL-1β is normally the mature circulating form. The cleavage of Pro-IL-1β to the mature form of IL-1β is catalyzed by caspase-1, which cleaves between Asp¹¹⁶-Ala¹¹⁷. Mature IL-1β is a 153 amino acid, 17.5 kDa, inflammatory cytokine that originated as residues 117 to 269 of Pro-IL-1β. However, under certain conditions a small quantity of Pro-IL-1β is also found extracellularly, where it is subject to non-specific cleavage at residues close to position 117 by proteases such as trypsin and elastase. The variant IL-1β forms produced by extracellular proteolytic cleavage vary in size. Some forms are fully active while others have only partial or no bioactivity. The Pro-IL-1β assay used to measure Pro-IL-1β in plasma samples from septic ICU patients was a colorimetric sandwich enzyme immunoassay (EIA). The “capture” antibody coated onto microtiter wells is specific for the Pro-piece of the molecule, i.e. the first 116 residues, and the “detection” antibody is specific for mature IL-β1. Thus, the assay does not detect either the Pro-piece alone or the mature IL-1β form alone—it is specific instead for the intact Pro-IL-1β.

b. The Pro-Interleukin-18 (Pro-IL-18) assay concerns Pro-IL-18 which is synthesized as a 193 amino acid long, 24 kDa inactive molecule that must be cleaved to produce the active mature cytokine. Pro-IL-18 has no known bioactivity. Mature IL-18 is an 18 kDa cytokine and is a co-stimulatory factor for production of interferon-β (IFN-γ). Caspase-1 cleaves (and thereby activates) Pro-IL-18 between Asp³⁶-Tyr³⁷ residues to produce the mature, bioactive cytokine that is readily secreted from cells. IL-18 is produced by activated macrophages, keratinocyts, intestinal epithelial cells, osteoblasts, adrenal cortex cells, Kupffer cells, and murine diencephalon. IL-18 acts on helper T type-1 (Th1) cells and in combination with IL-12, strongly induces Th1 cells to produce IFN-γ which plays a critical role in the defense against microbial pathogens. Pleiotropic effects of IL-18 have also been reported, such as, enhanced production of IFN-γ and GM-CSF in peripheral blood mononuclear cells (PBMCS), production of IL-2, GM-CSF and IFN-γ in T cells, enhanced expression of Fas ligand by Th1 cells, and increased production of Th1 cytokines. The serum/plasma assay used for Pro-IL-18 was a chemiluminescent sandwich enzyme immunoassay (EIA). The “capture” antibody binds to both human Pro-IL-18 and mature IL-18, but the “detection” mouse monoclonal antibody binds exclusively to the “Pro-region” of human Pro-IL-18, and does not cross-react with the mature IL-18 cytokine. Thus, the EIA is specific for human Pro-IL-18.

c. The Pro-Interleukin-33 (Pro-IL-33) assay concerns Human Pro-IL-33 which is 270 amino acids in length, is a 31 kDa member of the IL-1 family of proteins, and is a nuclear factor that also regulates gene transcription. Pro-IL-33 is constitutively expressed in smooth muscle and airway epithelia where it reportedly has two functions. First, it induces Th2-type cytokines, and second, it acts as a nuclear transcription factor. Pro-IL-33 contains a “Pro-region” (residues 1-111) and a mature cytokine carboxyl-terminal segment (residues 112-270). The “Pro-region” contains an α-helical homeodomain-like helix-turn-helix (HTH) DNA binding motif (residues 1-65), and a bipartite nuclear localization sequence (residues 61-78). The HTH motif mediates nuclear localization and heterochromatin association. The expression of Pro-IL-33 is upregulated in arterial smooth muscle, dermal fibroblasts, and keratinocyts following IL-1β induction. Pro-IL-33 is cleaved by caspase-1 between residues Ser¹¹¹-Ser¹¹² to yield the mature IL-33 cytokine which is released from the cells. Secreted IL-33 induces Th2 polarized lymphocytes to secrete IL-5 and IL-13, increases the production of IgA and IgE, and enhances inflammatory infiltration of mucosal tissues. The Pro-IL-18 assay used was a chemiluminescent, sandwich EIA that uses an affinity purified goat polyclonal IgG that is specific for the “Pro-region” as the “capture” antibody. A biotinylated affinity purified goat polyclonal IgG, that is specific for the carboxyl terminal mature IL-33 region, serves as the “detection” antibody, in the sandwich EIA.

Other blood biomarkers have been found to show aberrant apoptosis/secondary necrosis. Such biomarkers include co-induced enzymes. Again, in conjunction with a positive iNOS result from the IVD test of U.S. Pat. Nos. 6,581,578 and 7,198,904, the presence of these inducible stress related enzymes confirm that the patient is entering the sepsis pathology, for example:

a. Cyclooxygenase 2 (COX-2, also known as inducible COX) is a 72 kD enzyme that catalyzes the conversion of arachidonic acid to prostaglandin H2 (PGH2), the precursor of the 2-series prostanoids, which is the first step in the biosynthesis of prostaglandins (PGs), thromboxanes, and prostacyclins. Under normal conditions, COX-2 is undetectable in most cells and tissues, except in some specialized cells involved in reproduction, immunity, bone resporption, and pancreatic secretion. However, COX-2 is an inducible enzyme that becomes abundant in activated macrophages and other cells at sites of inflammation. COX-2 expression is induced by lipopolysaccharide (LPS), peptoglycan, and inflammatory cytokines, and was initially identified as an immediate-early growth response gene. COX-2 shares approximately 60% sequence homology with the constitutively expressed COX-1 enzyme which is a 70 kD protein that catalyzes the same enzymatic reaction. Both enzymes contain two active sites: a cyclooxygenase site, where arachidonic acid is converted into the hydroperoxy endoperoxide prostaglandin G₂ (PGG₂), and a heme site with peroxidase activity, that is responsible for the reduction of PGG₂ to PGH₂. Since iNOS and COX-2 are co-induced by the same stimuli, they are expressed in the same cells following induction. The rupture of induced cells that contain iNOS by secondary necrosis will lead to the presence of COX-2 in the blood. Thus, the presence of COX-2 is a confirmatory test for the processes of induction, aberrant apoptosis, and secondary necrosis which leads to the release of MV-A iNOS and ultimately to the sepsis pathology.

b. Hemoxygenase-1 (HO-1, which is also known as heat shock protein 32 (Hsp32)) is a ubiquitous soluble inducible stress-response enzyme that serves a vital metabolic function. It catalyzes the rate-limiting step in the heme degradation pathway and in the maintenance of iron homeostasis. HO-1 cleaves free heme into carbon monoxide, iron (which induces the expression of heavy-chain ferritin, an iron-sequestering protein), and biliverdin (which is converted to bilirubin by biliverdin reductase). Animal experiments have revealed a central role for HO-1 in tissue homeostasis, protection against oxidative stress and in the pathogenesis of certain diseases. The induction of HO-1 occurs in response to multiple forms of cellular stress, including exposure to LPS and inflammatory cytokines. The expression of HO-1 has been found to be induced in monocytes in patients with severe sepsis and septic shock. The detection and measurement of HO-1 in plasma of patients with Acute Respiratory Distress Syndrome (ARDS) has also been reported. Although unreported, since iNOS and HO-1 are co-induced by the same stimuli, they will be expressed in the same cells following induction. The rupture of induced cells that contain iNOS by secondary necrosis will lead to the presence of HO-1 in serum and plasma samples from septic patients. The presence of HO-1 was, thus, a confirmatory test for the processes of induction, aberrant apoptosis, and secondary necrosis which leads to the release of MV-A iNOS and, ultimately, to the sepsis pathology.

Indicators of organ damage or dysfunction maybe employed, for example:

a. Reg 1α (aka PSP=pancreatic stone peptide) is a very sensitive blood biomarker for organ damage caused by circulating plasma iNOS during an episode of sepsis. If plasma iNOS and plasma/serum Reg 1α are both present, then organ damage has already begun and, as the level of Reg 1α increases, the amount of organ damage has increased. It is, thus, an indicator of increasing internal organ damage, and places an individual patient into the “severe sepsis” stage. If plasma iNOS is present but plasma/serum Reg 1α is not present, then the patient's pathology is in the very early stages and organ damage is minimal or absent.

b. The L-Lactate assay is a plasma biomarker and is associated with hypoxia. The serum/plasma (blood level) concentration of this marker increases with organ/tissue damage. In a sepsis panel of tests, its appearance will coincide with the organ dysfunction that occurs in severe sepsis, and its blood level will increase as the patient deteriorates into septic shock. If effective treatment is applied, the blood level will decease as the patient improves, since the organ/tissue hypoxia, that results from hypoperfusion, resolves as the hemodynamic problems improve. Thus, an increase in L-lactate with a positive iNOS tests confirms the patient is septic and has organ damage. In other words, in this case, the patient was at least in severe sepsis and possibly in septic shock. A positive iNOS test in the absence of an increase in L-lactate indicates the patient is in the early stages of an episode of sepsis, since organ damage from hypoxia has not yet begun.

Blood indicators of continuing and/or increasing inflammation are also used in the present invention. One or more of these plasma/serum biomarkers can be employed to monitor the inflammatory state in an individual undergoing an episode of sepsis, for example

a. TNFalpha is an inflammatory cytokine that induces the expression of iNOS.

b. IL-1beta is an inflammatory cytokine that induces the expression of iNOS; and

c. IL-6 is an inflammatory cytokine that induces the expression of iNOS. If plasma iNOS is present and one or more of these inflammatory cytokines is present above normal plasma/serum levels, then the patient's condition will likely worsen.

d. C-reactive protein assay (CRP) is a plasma biomarker in the form of an acute phase protein. Such protein appears in blood samples relatively early in an episode of sepsis and other inflammatory conditions. It is associated with infections and many other inflammatory processes, since the release of various inflammatory cytokines caused by the infection leads to the expression of this acute phase protein. Its serum/plasma concentration increases as infections worsen and more inflammatory cytokines are released. However, it is not specifically indicative of the sepsis pathology, since many infections never deteriorate into the sepsis pathology (only˜15% of patients with confirmed infections progress to sepsis). Further, as an infection is successfully treated, the blood level of CRP drops even though the patient can be septic or may be entering the septic pathway due to the release of cell wall components from dead micro-organisms. In the iNOS test of U.S. Pat. Nos. 6,531,578 and 7,198,904, the appearance of iNOS will coincide with the early appearance of plasma CRP, and confirm the existence of an infection. Together, these tests will indicate the person is septic or is entering the septic pathway.

Blood indicators of the anti-inflammatory state are also found in the panel of the present invention. One or more of these plasma/serum biomarkers can be used to monitor the anti-inflammatory state in an individual undergoing an episode of sepsis to show they are recovering. For example:

a. IL-8 is an anti-inflammatory cytokine that is a negative predictor of sepsis; and

b. IL-10 is an anti-inflammatory cytokine that is a negative predictor of sepsis.

If the plasma level of iNOS is decreasing and one or more of these plasma/serum biomarkers of the anti-inflammatory state are present above normal levels, then the patient is most likely recovering from their episode of sepsis.

Blood biomarkers of primary and secondary necrosis are also found in the present invention. One or more of these plasma/serum proteins or protein complexes might be used to monitor ongoing secondary necrosis of apoptotic cells which leads to the release of circulation plasma iNOS and causes the sepsis pathology, for example:

a. LDH as a biomarker of both primary and secondary necrosis; and

b. Cytochrome C bound to its serum binding protein as a protein complex is a biomarker for ongoing secondary necrosis of apoptotic cells. If one or more of the inflammatory cytokines are present and the plasma/serum level of cytochrome C+CyC binding protein complex is also increasing, then induced iNOS-containing apoptotic cells are still undergoing secondary necrosis. This will lead to an increase in the circulating level of iNOS including microvesicle-associated iNOS and a worsening of that patient's condition.

It should be realized that each of the above named biomarkers have been tested individually as a biomarker for the sepsis pathology and found not to be useable. Individually, only the plasma iNOS test of U.S. Pat. No. 6,531,578 or 7,198,904 has been found to prognose the onset of sepsis; to diagnose sepsis by differentiating patients with inflammation from those with inflammation that will progress into hyperinflammation with organ damage and dysfunction, that are the hallmarks of an episode of sepsis; and to monitor the course of the sepsis pathology accurately. However, the supplementary biomarkers in the heretofore described panel of blood tests can provide valuable additional information to an attending physician, and serve as a foundation for a treatment decision tree. That is to say, based upon the presence or absence of iNOS in plasma and the blood levels of other biomarkers, a physician is critically assisted in deciding upon the best course of treatment for an individual patient.

Thus, if iNOS is not present in a patient's plasma but that patient's plasma/serum level of lactadherin is low, then such patient would be at a high risk of developing sepsis under any inflammatory conditions, such as major surgery. Since TNFalpha, IL-1beta, and IL-6 are released as part of the “cytokine storm” associated with major trauma, such as surgery, and since they, individually, and, in combination, induce the expression of iNOS in many different cell types, increased blood levels of these molecules heralds the expression of iNOS in many different cell types, increased blood levels of these inflammatory molecules heralds the onset of the inflammatory state and/or the hyperinflammatory state. With a low level of lactadherin and a high level of one or more of these inflammatory cytokines, an individual might develop sepsis very rapidly. If iNOS is present and increasing levels of the cytochrome C-binding protein complex are detected, there is a high likelihood that patient's condition will worsen as more induced, iNOS-containing apoptotic cells undergo secondary necrosis and release more iNOS, including MV-A iNOS, into the circulatory system. Similarly, if iNOS is present and increasing levels of the inflammatory cytokines, such as TNFalpha, IL-1beta and IL-6 are found, then that patient's condition will most likely worsen. Once the plasma level of iNOS starts to decrease and an increase in the plasma/serum level of the anti-inflammatory cytokines starts, then that patient is starting to recover and the treatment provided has been successful.

In summary, the presence of iNOS, including MV-A iNOS, in plasma samples has been found to be very highly associated with the onset of the sepsis pathology and with the organ damage and dysfunction that results from this hyperinflammatory condition. Thus, the presence or absence of plasma iNOS can be used as the central parameter in a decision tree in conjunction with the blood levels and with the increase or decrease in blood level of the other biomarkers in panel of blood tests of the present application. As heretofore stated, this panel of blood tests can provide an attending physician with very useful information on the status of their patients regarding the sepsis pathology and can help them decide upon the best course of treatment for their patients.

Table 1 represents a decision table using the panel of plasma biomarker heretofore discussed to determine the sepsis condition in a patient.

TABLE 1

| Plasma BioMarker | Test Results | Decision Output |
|---|---|---|
| iNOS | None present | Patient is non-septic |
| | Present above 1.5 | Patient is septic or |
| | ng/ml | is becoming septic |
| Lactadherin | Normal level and | Patient is not septic |
| | negative iNOS test | and less likely to |
| | | become septic |
| | Normal level and | Patient is septic and |
| | positive iNOS test | could improve with |
| | | aggressive |
| | | antimicrobial therapy |
| | Below normal range | Patient is at risk |
| | and negative iNOS | for developing sepsis |
| | test | |
| | Below normal and | Patient is septic and |
| | positive iNOS test | will likely deteriorate |
| Reg 1α | Normal level and | Patient is not septic |
| | negative iNOS test | and no organ damage |
| | Above normal level | Patient has ongoing |
| | and negative iNOS | organ damage and |
| | test | could be at risk for |
| | | the onset of sepsis |
| | Normal level and | Patient is in the |
| | positive iNOS test | very early stages of |
| | | the sepsis pathology |
| | Above normal level | Patient is septic and |
| | and positive iNOS | has organ damage, |
| | test | thus is in the severe |
| | | sepsis stage |
| HemeOxygenase 1 | Normal level and | Patient is not septic |
| (HO-1) (or COX-2 as a | negative iNOS test | and no secondary |
| possible substitute) | | necrosis is occurring |
| | Normal level and | Patient is septic but |
| | positive iNOS | secondary necrosis |
| | | has stopped |
| | Above normal level | Patient is not septic |
| | and negative iNOS | but has ongoing |
| | | inflammation and |
| | | necrosis and is high |
| | | risk for onset of |
| | | sepsis pathology |
| | Above normal level | Confirms sepsis, |
| | and positive iNOS | inflammatory |
| | | induction, and |
| | | secondary necrosis |
| | Decreasing HO-1 and | Patient is septic but |
| | positive iNOS | secondary necrosis is |
| | | decreasing |
| | Increasing HO-1 and | Patient is septic and |
| | positive iNOS | the sepsis pathology |
| | | is worsening |
| Pro-Interleukin-1β | None present and | Confirms patient is |
| (Pro-IL-1β) | negative iNOS test | not septic and no |
| (or Pro-IL-33 or Pro- | | secondary necrosis is |
| IL-18 as possible | | occurring |
| substitutes) | None present and | Patient is septic and |
| | positive iNOS test | confirms secondary |
| | | necrosis has stopped |
| | Present and negative | Patient is not septic |
| | iNOS test | but is confirmed to |
| | | have ongoing |
| | | inflammation and |
| | | necrosis and is high |
| | | risk for onset of |
| | | sepsis pathology |
| | Present and Positive | Confirms sepsis, |
| | iNOS test | inflammatory |
| | | induction, and |
| | | secondary necrosis |
| | Decreasing Pro-IL-1β | Patient is septic but |
| | and positive iNOS | confirms secondary |
| | test | necrosis is decreasing |
| | Increasing Pro-IL-1β | Patient is septic and |
| | and positive iNOS | confirms the sepsis |
| | | pathology is worsening |

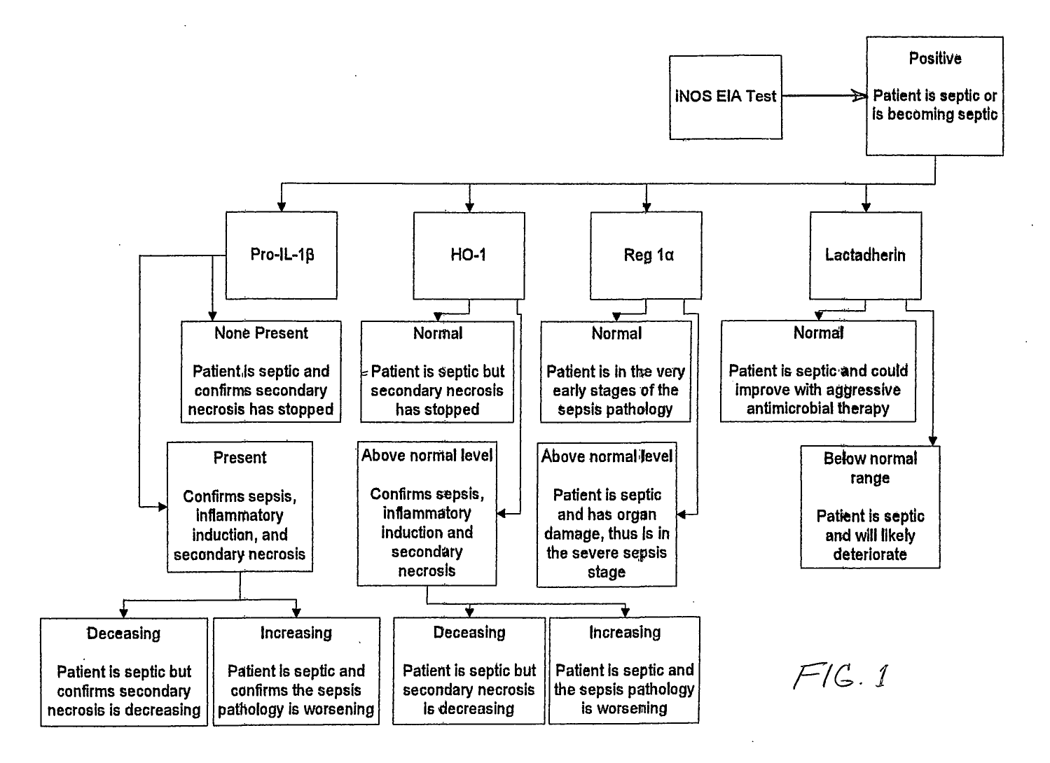

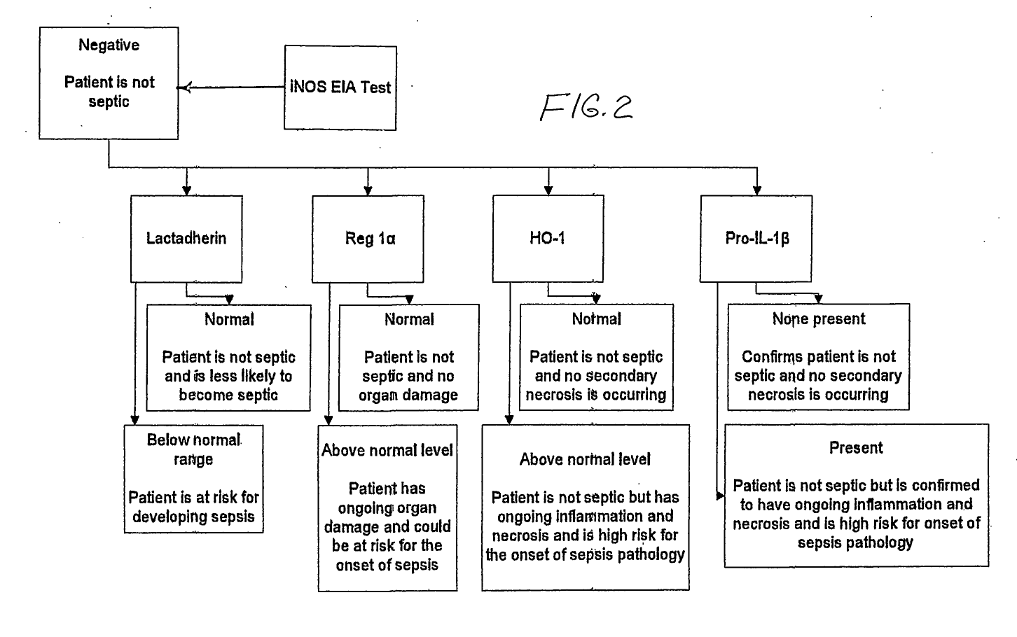

FIGS. 1 and 2 represent a decision tree which expresses the information of Table 1 in a flow diagram format.

Using Table 1 and FIGS. 1 and 2, an attending physician may decide upon a course for treatment for a patent with respect to sepsis conditions.

As a first demonstration, of the use of Table 1 and FIGS. 1 and 2, suppose a patient is iNOS negative, has a below normal level of plasma lactadherin, has an above normal plasma level of Pro-IL-1β. This would indicate the patient is currently not septic (since plasma iNOS was not detected) but is at a very high risk for the onset of the sepsis pathology since the patient's lactadherin level is low, the inflammatory pathway is active, and necrosis is occurring. This patient should be retested often over the next few days since sepsis could start at any moment.

As a second illustration of the use of Table 2 and FIGS. 1 and 2, suppose a patient is iNOS positive, has a normal level of plasma lactadherin, has a normal plasma level of Reg1α, has a normal plasma level of hemeoxygenase-1, and no plasma Pro-IL-1β is detected. This would indicate the patient is septic (since plasma iNOS is present) but is in the very early stages of the pathology. It would also indicate that secondary necrosis has stopped, been confirmed to have stopped, and that aggressive therapy would probably help. This patient should be treated with broad spectrum antibiotics even in the absence of a positive lab culture since it will take at least 24-48 hours for the lab culture results to be known. This patient should also be retested at least daily during the 7-10 day course of antibiotic therapy since the “cytokine storm” could be triggered by the cell wall fragments from dead bacteria.

While in the foregoing, embodiments of the present invention have been set forth in considerable detail for the purposes of making a complete disclosure of the invention, it may be apparent to those of skill in the art that numerous changes may be made in such detail without departing from the spirit and principles of the invention.

The following Examples are intended to further illustrate the invention sought for patenting, but are not intended to limit the scope of the invention herein.

### Example I

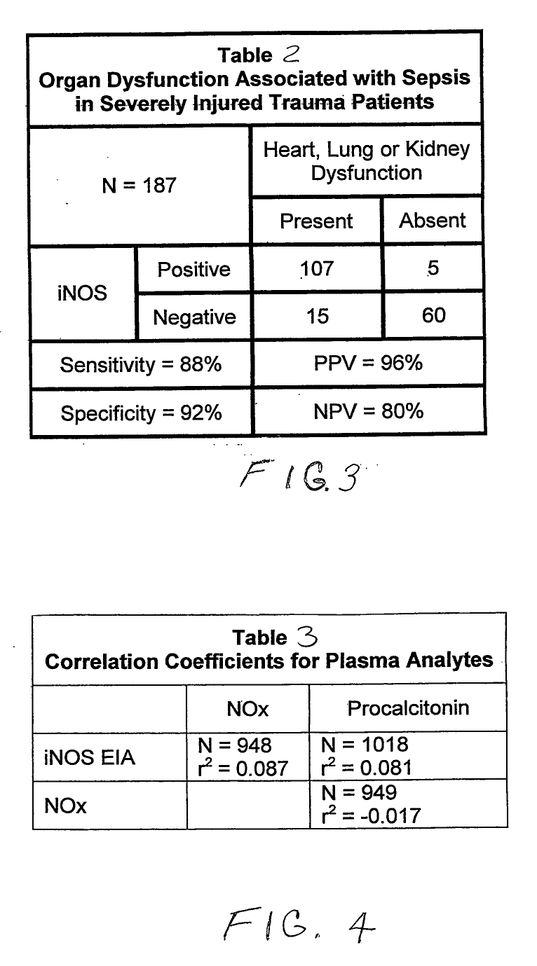

To date, three clinical studies focused on answering basic science questions have been conducted. A first pilot study on 10 ICU patients and 8 normal healthy volunteers; a second study on 47 ICU patients and 11 normal healthy volunteers; and a third clinical study on 238 ICU patients and 36 healthy volunteers took place. The novel discovery that the normally intracellular protein iNOS can be detected, using the diagnostic of U.S. Pat. No. 6,531,578, and measured in plasma was initially made on samples obtained from the 10 ICU patients enrolled in the first pilot clinical study. In this first pilot clinical study, iNOS was not detected in any of the plasma samples obtained from normal healthy volunteers who served as controls. In the three clinical studies combined, the onset of the sepsis pathology, as judged by the presence of iNOS in plasma samples, was detected 24-72 hours prior to the appearance of the physiological symptoms of sepsis in more than 65 ICU patients and prior to heart, lung, and kidney dysfunction in more than 100 severely injured trauma patients, as shown in Table 2 of FIG. 3.

### Example II

An analysis of the data obtained from the trauma patients and normal healthy volunteer controls, who were enrolled in the third clinical study of EXAMPLE I, concerning the IVD test of U.S. Pat. Nos. 6,531,578 and 7,198,904, was performed. The analyses focused on predicting hemodynamic, pulmonary, and renal dysfunction associated with the sepsis pathology in trauma patients (Table 3 of FIG. 4). These analyses determined the predictive value of such IVD test where:

[1] Hemodynamic dysfunction was defined as mean arterial pressure (MAP)≦70 mm Hg or the patient was receiving one or more pressor drugs;

[2] Pulmonary dysfunction was defined as a diagnosis of respiratory failure or mechanical ventilation for >24 hours or SIMV with changes in blood gasses and pH; and

[3] Renal dysfunction was defined as blood urea nitrogen (BUN)>20 mg/d1.

The results of these analyses showed that the plasma iNOS IVD test of U.S. Pat. Nos. 6,531,578 and 7,198,904 had a positive predictive value (PPV) of greater than 96% and a negative predictive value (NPV) of 80% for organ dysfunction associated with sepsis in trauma patients. Of special note, both the positive predictive value and the negative predictive value as well as the sensitivity and specificity values for such plasma iNOS assay might have been higher if other considerations are also taken into account. For example, of the five patients where iNOS was detected in their plasma, but were not yet suffering from heart, lung or kidney dysfunction, iNOS was only detected in their blood sample at the end of the 5 day study period. It is theorized that they may have become septic and suffered organ dysfunction after the study period ended. Similarly, of the 15 patients who had organ dysfunction, but were plasma iNOS negative throughout the study period, all had suffered traumatic injuries that affected one of more of their major organs. Thus, organ dysfunction might well be expected—not due to the sepsis pathology, but due to their serious injuries.

### Example III

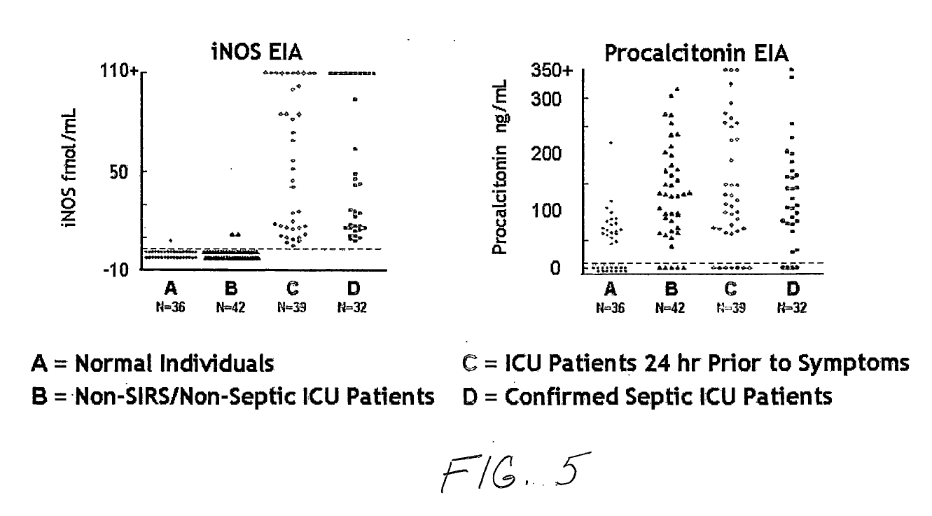

The degree of correlation between plasma iNOS and other potential biochemical markers of sepsis was determined using the data collected in the third clinical study of EXAMPLE I. The plasma levels of NOx (which is the combination of plasma nitrate plus nitrite, the two breakdown products of nitric oxide), and procalcitonin were also measured in addition to plasma iNOS. No correlation between plasma iNOS and plasma nitrate plus nitrite (NOx) was found. This is believed to demonstrate that iNOS in plasma is not an active enzyme, since plasma does not contain two of its required co-factors for enzymatic activity, Table 3 of FIG. 4. Thus, there is no nitric oxide produced by the circulating iNOS to breakdown to nitrate or nitrite. The plasma level of procalcitonin, an FDA approved IVD test for diagnosing sepsis, did not correlate with the sepsis pathology (Table 3 of FIG. 4 and the iNOS EIA and procalcitonin EIA test results of FIG. 5). Only the detection of plasma iNOS both forecast the onset of sepsis and mirrored the course of the pathology correlated with the sepsis pathology.

### Example IV

Four cohorts from the third clinical study of EXAMPLE I were analyzed for the plasma levels of iNOS and procalcitonin, an FDA approved IVD test for sepsis, as potential biomarkers for the sepsis pathology (FIG. 5, plasma iNOS [iNOS EIA] and plasma procalcitonin [Procalcitonin EIA]. The four cohorts depicted in both of the scatter diagrams of FIG. 5 are as follows: Group A comprise the 36 normal healthy individuals (normal); Group B comprise the ICU patients who remained non-SIRS/non-septic throughout the entire study period; Group C comprise the ICU patients who became septic during the study period, and their plasma levels are shown 24 hours before the symptoms of sepsis were recognized by the attending doctors; and Group D comprise the confirmed septic patients who were enrolled in the study and had not received antibiotics prior to enrollment. The cut-off values for the normal plasma levels of the biomarkers are depicted by a dashed line (- - - - - -) Only plasma iNOS was found to differentiate between non-septic trauma patients (group B) and those individuals who are developing sepsis (group C), or who/were already septic (group D). The plasma level of procalcitonin, one of the FDA approved IVD tests, was not specific for sepsis, since a number of normal healthy volunteers and many of the non-SIRS/non-septic trauma patients (groups A and B) had elevated plasma levels of procalcitonin.

### Example V

### Plasma iNOS levels

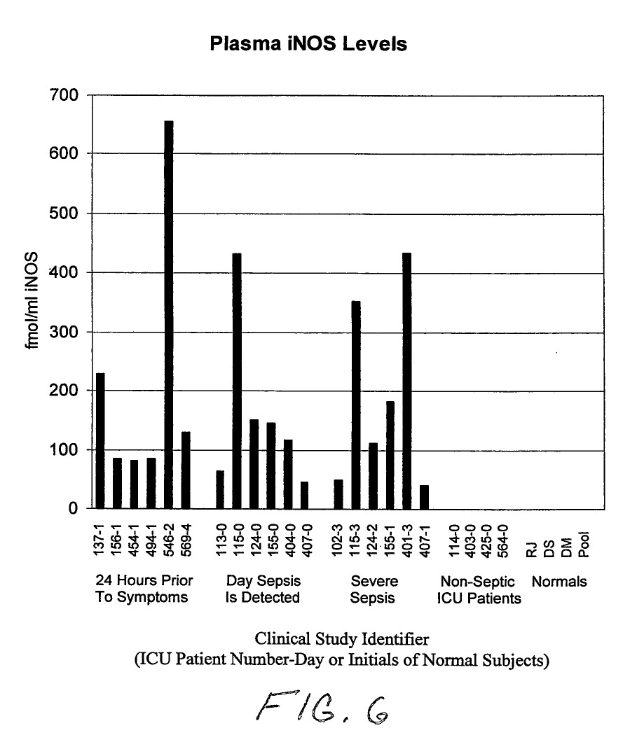

Plasma samples were selected from banked frozen samples obtained during the third clinical study of EXAMPLE I on plasma iNOS as a potential new biomarker for the sepsis pathology. The samples were selected based upon a number of criteria including the amount of plasma still remaining as frozen banked plasma since the iNOS test plus the other tests to be performed on the plasma would require at least 1.8 ml of plasma to complete all the assays. The samples were also selected based upon the characteristics of the person from whom the sample was obtained and the stage of the pathology the individual was at when the sample was collected: (1) early in the sepsis pathology, i.e. at least 24 hours before the symptoms of sepsis appeared, (2) on the day sepsis was confirmed by a positive lab culture, (3) on the day when the ICU patient became severely septic, i.e. the day the attending physicians recognized the beginning of organ dysfunction associated with the sepsis pathology, (4) ICU patients who remained non-septic throughout the entire study period, and (5) normal individuals including a pool of plasma obtained from 10 healthy individuals, FIG. 6.

Of the biomarkers tested, only plasma iNOS has been found to be specific for the sepsis pathology since it was (1) the only biomarker present in all of the plasma samples obtained from septic patients and from the plasma of all the ICU patients who would become septic in the next 24-72 hours and (2) the only biomarker absent from the plasma of all the non-septic ICU patients and all the normal human samples. While plasma iNOS solely by itself can serve as a good biomarker for the onset of the sepsis pathology and can be used to monitor the course of an episode of sepsis, additional information regarding an individual patient's status would be of utility to the attending physicians. Thus, additional biomarkers were deemed to be needed for the analysis of the sepsis pathology in order to form a panel of biomarkers that can supplement and extend the information the plasma iNOS test is providing to the attending physicians.

### Example VI

### Plasma Lactadherin Levels

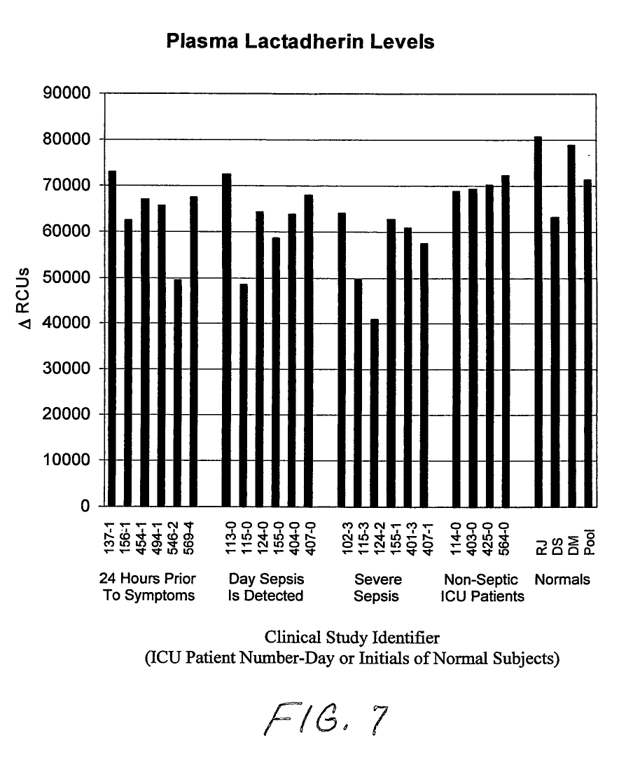

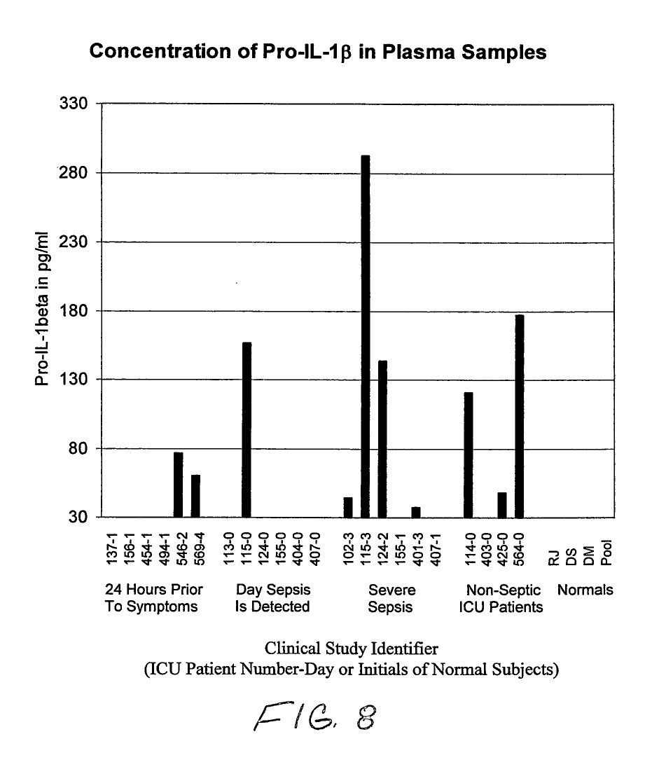

Employing the sample selection process of EXAMPLE V, the levels of plasma lactadherin were ascertained, FIG. 7.

The relative plasma level of lactadherin, which is also known as milk fat globule epidermal growth factor-factor VIII (MFG-E8) and as breast antigen-46(BA46), was determined using a sandwich EIA. Two aliquots of each plasma sample were assayed: one aliquot was stock plasma, and the other aliquot was immunodepleted plasma which had been incubated for 60 minutes with the capture antibody immobilized onto the side of a plastic microtiter well. The difference, i.e. delta (Δ), in the intensity of the sandwich EIA readout as Relative Chemiluminescent Units (RCUs) provided a relative measure of the amount of lactadherin immunodepleted from the pre-incubated sample. The average RCUs removed during the immunodepletion incubation for the normal subjects and for the non-septic patients is 71, 800±5,300 RCUs. Patient samples 546-2 (68.9% of normal), 115-0 (67.7% of normal), 115-3 (68.7% of normal) and 124-2 (57.0% of normal) had lactadherin levels more than three standard deviations less than the normal plasma level. Similarly, patient samples 155-0 (81.4% of normal), 401-3 (84.8% of normal) and 407-1 (79.9% of normal) had plasma lactadherin levels more than two standard deviations less than normal plasma levels. Further, as patients progressed from sepsis to severe sepsis, their plasma lactadherin level either remained depressed or deceased even further from the normal level. Thus, in conjugation with a plasma iNOS assay, lactadherin was shown to be a valuable biomarker (1) for assessing an individual's susceptibility to become septic and (2) for monitoring an individual patient's deterioration as the sepsis pathology progresses, as is illustrated by patients #115, 124, 155 and 407, who all progressed from septic to severely septic during the study period.

### Example VII

### Pro Interleukin-1β Concentrations

Employing the sample selection process of EXAMPLE V, the concentrations of plasma Pro-IL-1β were ascertained, FIG. 8.

The plasma concentration of Pro-Interleukin-β (Pro-IL-1β) was measured using a commercially available EIA (R&D Systems catalogue #DLBPOO) exactly as described in the kit instruction manual except the readout at the end of the assay used OPD/H₂O₂ instead of TMB/H₂O₂. The presence of ProIL-1β was not specific for the onset of sepsis since it was elevated above normal levels in many of the non-septic patients. However, a trend towards high quantities was observed as the sepsis pathology worsened from confirmed sepsis to severe sepsis with organ dysfunction. It was determined that Pro-IL-1β can serve as a confirmatory test for the presence of and continuation of aberrant apoptosis that turns into secondary necrosis since, under normal conditions, Pro-IL-1β is cleaved prior to secretion from a cell to yield the mature cytokine IL-1β from which the “Pro” amino-terminal sequence has been cleaved. Thus, only under abnormal conditions, such as aberrant apoptosis that turns into secondary necrosis, would the pro-form of the cytokine be released into the circulatory system.

### Example VIII

### Pro-Interleukin-18 Levels

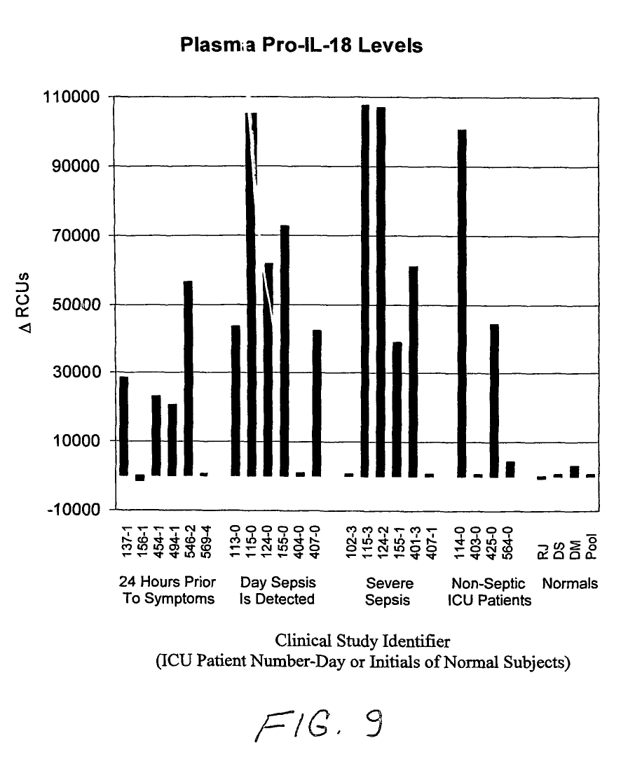

Employing the sample selection process of Example V, the levels of plasma Pro-IL-18 were ascertained, FIG. 9.

Pro-Interleukin-18 (Pro-IL-18) is the intracellular “pro-form” of the mature interleukin-18 (IL-18) cytokine. Pro-IL-18 is normally cleaved during the secretion process to yield the mature cytokine, thus circulating Pro-IL-18 only occurs when the cell dies by necrosis which results in the release of its cellular contents into the circulatory system. The relative plasma level of Pro-IL-18 was determined using a chemiluminescent sandwich enzyme-immunoassay (EIA). Two aliquots of each plasma sample were assayed: one aliquot was stock plasma, and the other aliquot was immunodepleted plasma which had been incubated for 60 minutes with the capture antibody immobilized on a microtiter well. The difference, i.e. delta (Δ), in the intensity of the sandwich EIA readout as Relative Chemiluminescent Units (RCUs) provided a relative measure of the amount of Pro-IL-18 immunodepleted from the pre-incubated samples. The average RCUs removed during the immunodepletion incubation for the normal subjects was 980 RCUs. Many of the ICU patients tested had elevated levels of plasma Pro-IL-18 that were significantly higher than the normal plasma samples tested. However, an increase in plasma Pro-IL-18 was not specific for the sepsis pathology, but can be used in conjugation with other plasma tests to indicate the occurrence of aberrant apoptosis of induced cells are occurring via secondary necrosis, and, thus, confirmed a positive plasma test for iNOS and the onset of the sepsis pathology. Pro-IL-18 was concluded to be a substitute for the preferred procytokine, Pro-IL-13 of EXAMPLE VII.

### Example IX

### Pro-Interleukin 33 Levels

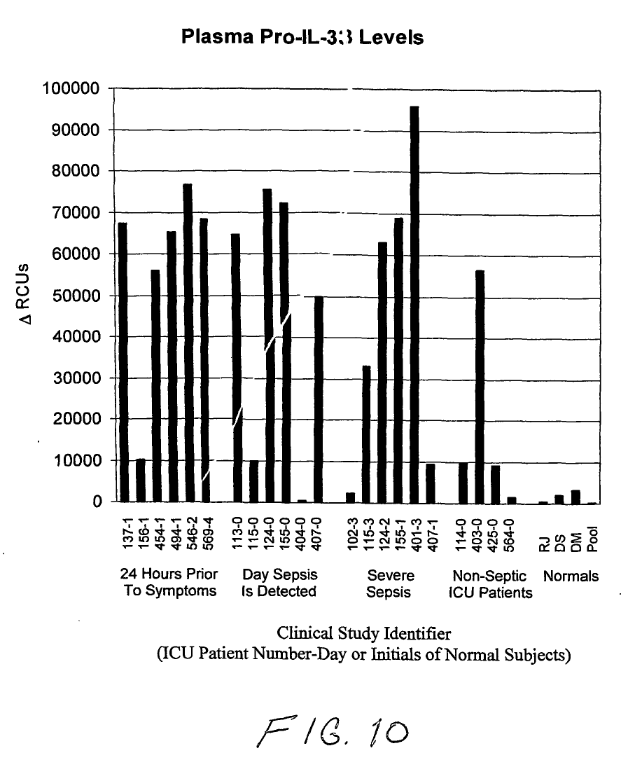

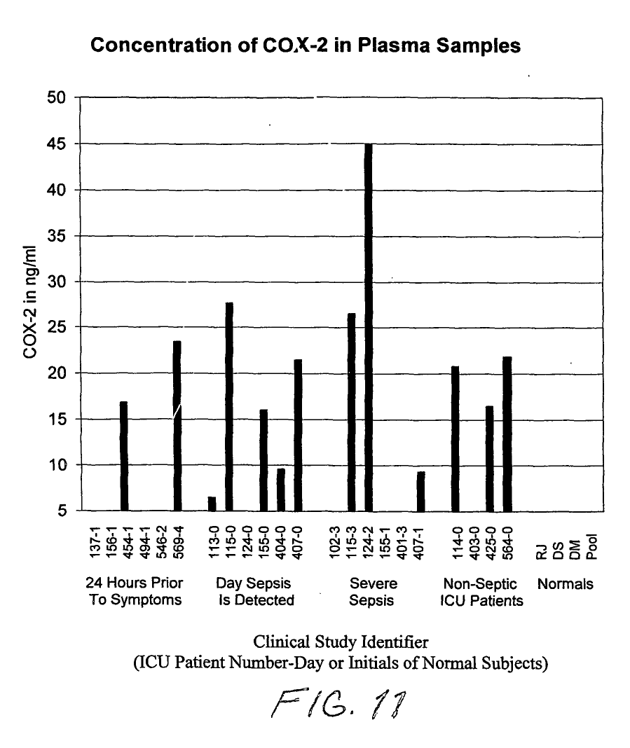

Employing the sample selection process of EXAMPLE V, the levels of plasma Pro-IL-33 were ascertained FIG. 10.

Pro-Interleukin-33 (Pro-IL-33) is the intracellular “pro-form” of the mature interleukin-33 (IL-33) cytokine. Pro-IL-33 is normally cleaved during the secretion process to yield the mature cytokine, thus circulating Pro-IL-33 only occurs when cells die by necrosis which results in the release of their cellular contents into the circulatory system. The relative plasma level of Pro-IL-33 was determined using a chemiluminescent sandwich enzyme-immunoassay (EIA). Two aliquots of each plasma sample were assayed: one aliquot was stock plasma, and the other aliquot was immunodepleted plasma which had been incubated for 60 minutes with the capture antibody immobilized on a microtiter well. The difference, i.e. delta (Δ), in the intensity of the sandwich EIA readout as Relative Chemiluminescent Unites (RCUs) provided a relative measure of the amount of Pro-IL-33 immunodepleted from the pre-incubated sample. The average RCUs removed during the immunodepletion incubation for the normal subjects was 1480 RCUs. Many of the ICU patients tested had elevated levels of plasma Pro-IL-33 that were significantly higher than the normal plasma samples tested. However, an increase in plasma Pro-IL-33 was not found exclusively in septic patients since two of the four non-septic ICU patients tested also had significantly elevated plasma levels of Pro-IL-33 as compared to normal individuals. Thus, Pro-IL-33 was not specific for the sepsis pathology, but was used in conjugation with other plasma tests to indicate aberrant apoptosis of induced cells is occurring via secondary necrosis, and thus, confirm a positive plasma test for iNOS and the onset of the sepsis pathology. However, Pro-IL-1β of EXAMPLE VII was the preferred confirmatory test for use in the sepsis testing panel, of the present invention.

### Example X

### Cyclooxygenase-2 Levels

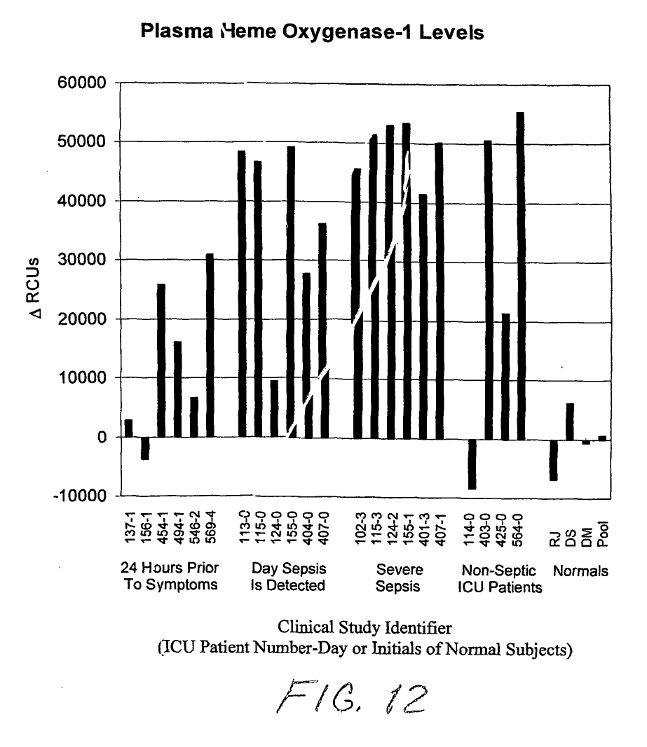

Employing the sample selection process of EXAMPLE V, the levels of plasma COX-2 were ascertained, FIG. 11.

Cyclooxygenase-2 (COX-2), also known as inducible cyclooxygenase, is an inducible microsomal enzyme that catalyzes the synthesis of prostaglandins from arachidonic acid. The plasma level of COX-2 was determined using a commercially available sandwich ELIS kit (Calbiochem Catalogue #CBA053) exactly as described in the kit instruction manual except the readout at the end of the assay used OPD/H₂O₂ instead of TMB/H₂O₂. As expected, the presence of COX-2 was not specific for the onset of sepsis since it was elevated above normal levels in many of the non-septic patients. While COX-2 was not specific for the hyperinflammatory sepsis pathology, an elevated plasma level of COX-2, in conjunction with a positive plasma iNOS test, confirmed that aberrant apoptosis of induced cells had turned into secondary necrosis and the secondarily necrotic cells were releasing their cellular contents into the circulatory system. Of the two co-inducible proteins studies, plasma HO-1 was the preferred analyte for inclusion in the sepsis test panel of the present invention but COX-2 could be used as a substitute.

### Example XI

### HEME Oxygenase-1 Levels

Employing the sample selection process of EXAMPLE V, the levels of plasma HO-1 were ascertained, FIG. 12.

HemeOxygenase-1 (HO-1), also known as Heat-Shock Protein-32 (HSP-32), is an inducible microsomal enzyme that cleaves heme to produce biliverdin, iron and carbon monoxide (CO). The relative plasma level of HO-1 was determined using a chemiluminescent sandwich enzyme-immunoassay (EIA). Two aliquots of each plasma sample were assayed: one aliquot was stock plasma, and the other aliquot was immunodepleted plasma which had been incubated for 60 minutes with the capture antibody immobilized on a microtiter well. The difference, i.e. delta (Δ), in the intensity of the sandwich EIA readout as Relative Chemiluminescent Units (RCUs) provided a relative measure of the amount of HO-1 immunodepleted from the pre-incubated sample. While HO-1 was not specific for the hyperinflammatory sepsis pathology as was shown by the occurrence of high levels in the plasma of non-septic ICU patients, in conjunction with a positive plasma iNOS test, an elevated plasma level of HO-1 confirmed that aberrant apoptosis of induced cells had turned into secondary necrosis and the secondarily necrotic cells were releasing their cellular contents into the circulatory system, confirming the positive plasma iNOS test. Further, as patients deteriorated and progressed from early sepsis to culture confirmed sepsis to severe sepsis with organ dysfunction, a trend towards an increase in the plasma concentration of hemeoxygenase-1 occurred as is illustrated by the RCUs increasing from 13,100 to 36,300 to 49,300, respectively. Plasma HO-1 was the preferred analyte for inclusion in the sepsis test panel of the present invention, but COX-2 of EXAMPLE X could be used as a substitute.

### Example XII

### REG 1α levels

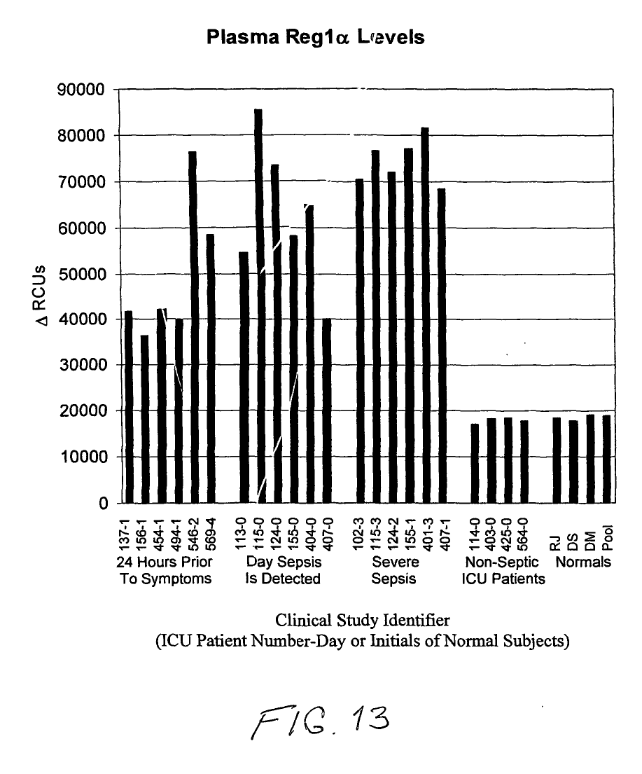

Employing the sample selection of EXAMPLE V, the levels of plasma Reg1α were ascertained, FIG. 13.

The relative plasma level of Reg1α, which is also known as Pancreatic Stone Peptide (PSP), was determined using a chemiluminescent sandwich enzyme-immunoassay (EIA). Two aliquots of each plasma sample were assayed: one aliquot was stock plasma, and the other aliquot was immunodepleted plasma which had been incubated for 60 minutes with the capture antibody immobilized on a microtiter well. The difference, i.e. delta (A), in the intensity of the sandwich EIA readout as Relative Chemiluminescent Units (RCUs) provided a relative measure of the amount of Reg1α immunodepleted from the pre-incubated sample. The average RCUs removed during the immunodepletion incubation for the normal subjects and for the non-septic ICU patients was 18,300±700 RCUs. All 18 of the septic ICU patient samples had elevated Reg1α levels that were more than four standard deviations higher than the normal plasma level. Further, as patients progressed from early sepsis to culture confirmed sepsis to severe sepsis with organ dysfunction, their plasma Reg1α level tended to increase even further above the normal level. Thus, in conjugation with a plasma iNOS assay, Reg1α was a valuable biomarker (1) for assessing organ damage very early during an episode of sepsis (even prior to the onset of the symptoms of sepsis) and (2) for monitoring an individual patient's deterioration as the sepsis pathology progressed, as is illustrated by patients #115, 124, 155, and 407 who all progressed from being septic to being severely septic during the study period.

### Example XIII

### C-Reactive Protein Concentrations

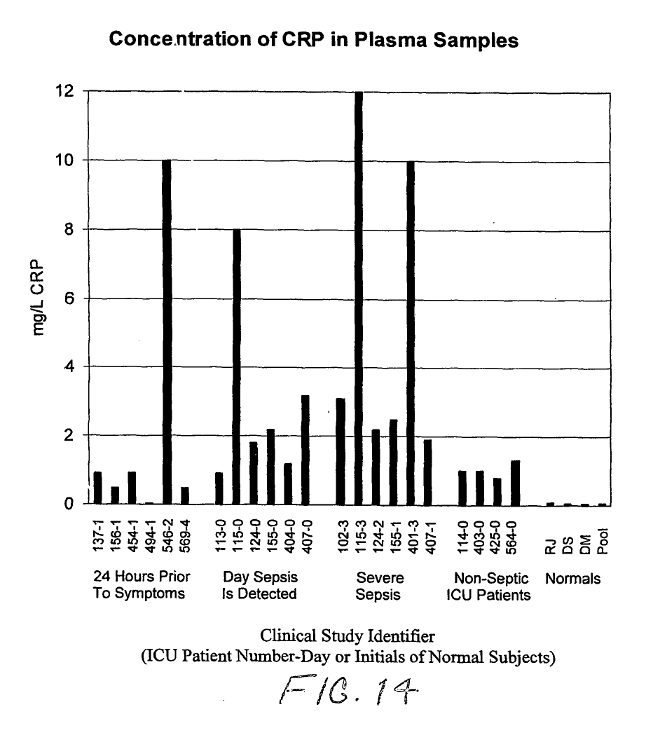

Employing the sample selection process of EXAMPLE V, the concentrations of CRP were ascertained, FIG. 14.

The plasma concentration of C-Reactive Protein (CRP) was measured using a commercially available EIA (BioCheck catalogue #BC-1119) exactly as described in the kit instruction manual except the readout at the end of the assay used OPD/H₂O₂ instead of TMB/H₂O₂. As expected, the presence of CRP was not specific for the onset of sepsis since it was elevated above normal levels in many of the non-septic patients. However, a trend towards higher quantities of CRP was observed as the sepsis pathology worsened from confirmed sepsis to severe sepsis with organ dysfunction. All but one of the ICU patients tested had plasma CRP levels elevated as compared to the normal population.

What is claimed:

1. An assay for assessing a sepsis condition in a human subject, comprising:
a. a test for determining the presence of iNOS in the plasma of the human subject; and
b. a test for determining the onset of a sepsis condition in the human subject.

2. The assay of claim 1 in which said test for determining the presence of iNOS in the plasma of the human subject comprises a monoclonal antibody recognizing iNOS.

3. The assay of claim 2 in which said test for determining the onset of a sepsis condition comprises a test for ascertaining the level of pro-cytokine in the plasma of the human subject.

4. The assay of claim 3 in which said pro-cytokine is selected from the group comprising: Pro-IL-1β, Pro-IL-18, and Pro-IL-33.

5. An assay for assessing a sepsis condition in a human subject, comprising:
a. a test for determining the presence of iNOS in the plasma of the human subject; and
b. a test for determining aberrant apoptosis/secondary cell necrosis of the human subject.

6. The assay of claim 5 in which said test for determining the presence of iNOS in the plasma comprises a monoclonal antibody recognizing iNOS.

7. The assay of claim 6 in which said test for determining aberrant apoptosis and secondary cell necrosis of the human subject comprises a test for ascertaining the level of an inducible stress related enzyme in the plasma of the human subject.

8. The assay of claim 7 in which said inducible stress related enzyme is selected from the group comprising: HO-1, and COX-2.

9. An assay for assessing a sepsis condition in a human subject, comprising:
a. a test for determining the presence of iNOS in the plasma of the human subject; and
b. a test for determining organ damage or dysfunction of the human subject.

10. The assay of claim 9 in which said test for determining the presence of iNOS in the plasma of the human subject comprises a monoclonal antibody recognizing iNOS.

11. The assay of claim 9 in which said test for determining organ damage or dysfunction of the human subject comprises a test for ascertaining the presence of a biomarker selected from the group comprising Reg1α and L-Lactate.

12. An assay for assessing a sepsis condition in a human subject comprising:
a. a test for determining the presence of iNOS in the plasma of the human subject; and
b. a test for determining continuing or increasing inflammation in a human subject.

13. The assay of claim 12 in which said test for determining the presence of iNOS comprises a monoclonal antibody recognizing iNOS.

14. The assay of claim 12 in which said test for determining continuing or increasing inflammation in a human subject comprises a test for ascertaining the presence of biomarkers selected from the group comprising:
TNFalpha, IL-1β, IL-6, and CRP.

15. An assay for assessing the predisposition to a sepsis condition in a human subject comprising:
a. a test for determining the presence of iNOS in the plasma of the human subject; and
b. a test for determining the presence of lactadherin in the plasma of the human subject.

16. The assay of claim 1 which additionally comprises: a test for determining aberrant apoptosis/secondary cell necrosis of the human subject

17. The assay of claim 16 which additionally comprises: a test for determining organ damage or dysfunction of the human subject.

18. The assay of claim 17 which additionally comprises: a test for determining continuing or increasing inflammation in a human subject.

19. The assay of claim 18 which additionally comprises: a test for determining the presence of lactadherin in the plasma of the human subject.

20. A decision tree system for assessing sepsis conditions in a human subject, comprising:
a. a test for determining the presence of iNOS in the plasma of the human subject; and
b. a test for determining the presence of a blood biomarker in the plasma of the human subject, said blood biomarker selected from the group consisting of: lactadherin, reg 1α, cyclooxygenase 2, hemeoxygenase-1, Pro-Interleukin 1β, Pro-Interleukin 33, and Pro-Interleukin-18, or a combination, thereof.
